(19) United States
(12) Patent Application Publication
(10) Pub. No.: US 2024/0359038 A1
(43) Pub. Date: Oct. 31, 2024

(54) ULTRASONIC TREATMENT DEVICE WITH LARGE FOCUSED AREA AND ULTRASONIC TREATMENT TIP

(71) Applicant: SHENZHEN PENINSULA MEDICAL GROUP, Shenzhen (CN)

(72) Inventors: Yanan LI, Shenzhen (CN); Yujia PENG, Shenzhen (CN); Xingli LI, Shenzhen (CN); Xiaobing LEI, Shenzhen (CN); Yi DING, Shenzhen (CN)

(73) Assignee: SHENZHEN PENINSULA MEDICAL GROUP, Shenzhen (CN)

(21) Appl. No.: 18/771,061

(22) Filed: Jul. 12, 2024

Related U.S. Application Data
(63) Continuation-in-part of application No. PCT/CN2024/078149, filed on Feb. 22, 2024.
(63) Continuation-in-part of application No. PCT/CN2023/091816, filed on Apr. 28, 2023.

(30) Foreign Application Priority Data
May 5, 2022 (CN) 202221058921.X
Jun. 25, 2023 (CN) 202310757822.3

(51) Int. Cl. A61N 7/02; A61N 7/00

(52) U.S. Cl. CPC A61N 7/02; A61N 2007/0008; A61N 2007/006; A61N 2007/0078; A61N 2007/027

(57) ABSTRACT

An ultrasonic treatment device with a large focused area includes: a housing; and an ultrasonic transducer installed inside the housing. Ultrasonic waves emitted by the ultrasonic transducer being configured to form a plurality of focused area units at one or more focused depths in a focused area, and each of the focused area units including one or more of a focused point, a focused line, a focused plane or a focused volume.

### CROSS-REFERENCE TO RELATED APPLICATIONS

The present application is a continuation-in-part of International Application No. PCT/CN2023/091816, filed on Apr. 28, 2023, which claims priority to Chinese Patent Application No. 202221058921.X, filed on May 5, 2022; and also a continuation-in-part of International Application No. PCT/CN2024/078149, filed on Feb. 22, 2024, which claims priority to Chinese Patent Application No. 202310757822.3, filed on Jun. 25, 2023. The disclosures of the above-mentioned applications are incorporated herein by reference in their entireties.

### TECHNICAL FIELD

The present application relates to the technical field of ultrasonic treatment equipment, and in particular to an ultrasonic treatment device with a large focused area and an ultrasonic treatment tip.

### BACKGROUND

In recent years, non-invasive beauty technology using a high-intensity focused ultrasound has been widely used. The principle is that the focused ultrasound ceramic wafer installed inside the treatment tip emitting focused ultrasound wave. After being conducted through the liquid conductor, the focused ultrasound wave is focused on the subcutaneous tissue or fat tissue, and forms a “high temperature point” with a very small volume (usually less than 1 mm) and a temperature ranging from 50° C. to 75° C. on the local subcutaneous tissue or fat tissue, so that the local subcutaneous tissue (skin tissue or fat tissue at a depth ranging from 1 mm to 18 mm) shrinks under the action of high temperature, and the fat tissue will perform “burning fat”, thereby achieving the beauty effect of improving skin firmness and losing weight.

The existing ultrasonic treatment device adjusts the frequency of ultrasonic emission on the basis of increasing the treatment area, so that the ultrasonic wave is focused to form a focused area along the horizontal or vertical direction. However, the focused area in the horizontal or vertical direction will act on the subcutaneous tissue layer of different depths of the human body, and the treatment effect of the subcutaneous tissue layer at different depths is uneven, resulting in the ultrasonic treatment device with the large focused area being unable to accurately treat the subcutaneous tissue layer at the target depth, so that the ultrasonic treatment device with the large focused area needs more time to effectively act on the subcutaneous tissue layer at the target depth, which greatly affects the treatment efficiency of the ultrasonic treatment device with the large focused area.

In the existing ultrasonic treatment tip, the ultrasonic ceramic wafer is usually provided in a sealed container, and the sealed container is filled with the liquid that can conduct ultrasonic waves. One side of the sealed container is made of a soft plastic hard thin acoustic film. The ultrasonic energy is emitted from the plastic hard thin acoustic film to the surface of the container, and the plastic hard thin acoustic film is pressed against the skin surface that has been coated with an ultrasonic coupling agent for improving a transfer of the ultrasonic energy. The ultrasonic wave can enter the action site of the subcutaneous tissue through the skin surface to act on the subcutaneous tissue. However, in the treatment of the existing ultrasonic treatment tip, the ultrasonic waves are usually focused at one point, the treatment area is small, and the efficiency is low.

### SUMMARY

The main purpose of the present application is to provide an ultrasonic treatment device with the large focused area and an ultrasonic treatment tip, aiming to form a focused area in the horizontal direction and accurately treat the subcutaneous tissue layer at a target depth, thereby improving the treatment efficiency of the ultrasonic treatment device with the large focused area and solving the problems of small treatment area and low efficiency of the ultrasonic treatment tip in the related art.

In view of the above objectives, the present application provides an ultrasonic treatment device with a large focused area, including:
a housing; and
an ultrasonic transducer installed inside the housing, ultrasonic waves emitted by the ultrasonic transducer being configured to form a plurality of focused area units at one or more depths in a focused area, and each of the focused area units including one or more of a focused point, a focused line, a focused plane or a focused volume.

In an embodiment, the plurality of focused area units are disposed in a rectangular array, a circular array, an elliptical array or a circular array.

In an embodiment, each of the focused point, the focused line and the focused volume has a corresponding heat diffusion zone.

In an embodiment, central temperatures of the plurality of focused area units are the same, and the central temperature of each focused area unit is in an area from 48° C. to 80° C. In an embodiment, central temperatures of two adjacent focused area units are different.

In an embodiment, shapes of the plurality of focused areas are the same, and each focused area is in a rectangular shape, a circular shape, an elliptical shape or an annular shape.

In an embodiment, shapes of the plurality of focused area units are different, and each focused area is in a rectangular shape, a circular shape, an elliptical shape or an annular shape.

In an embodiment, the ultrasonic transducer includes a concave-shaped emitting surface for focusing an ultrasonic wave beam, and
the concave-shaped emitting surface includes a plurality of emitting surface units disposed in an interval array; the plurality of emitting surface units are disposed in a concave array, and the plurality of emitting surface units have different curvature radius.

In an embodiment, the ultrasonic transducer includes a plurality of sub-transducer arrays, and the plurality of sub-transducers include a plurality of different focused depths, and are configured to form a plurality of focused area units at at least two focused depths.

In an embodiment, the ultrasonic transducer includes a central transducer and at least one sub-transducer, and the sub-transducer is provided adjacent to the central transducer and is provided outside the central transducer; the central transducer is a spherical transducer, and the sub-transducer is an annular transducer.

In an embodiment, the plurality of sub-transducers include a plurality of tile-shaped transducers disposed in an array; the plurality of tile-shaped transducers are configured to form a plurality of focused area units at the same focused depth, and the focused area units include focused lines.

In an embodiment, the plurality of focused area units are disposed at equal intervals, and/or a distance between two adjacent focused areas are in an area from 0.1 mm to 2 mm.

In an embodiment, the ultrasonic transducer includes a plane-shaped emitting surface and a concave-shaped acoustic lens for focusing an ultrasonic wave beam; the acoustic lens includes small lenses with different curvature radius, and the small lenses are independent with each other.

In an embodiment, the ultrasonic transducer includes an emitting surface and an ultrasonic treatment tip, and the emitting surface is plane-shaped; the ultrasonic treatment tip further includes an acoustic lens, and the acoustic lens is a curve-shaped acoustic lens; the acoustic lens is installed at the emitting surface, and an ultrasonic wave is refracted from the curve-shaped acoustic lens at at least two focused depths underneath human subcutaneous tissue to form a three-dimensional focused area.

In an embodiment, the ultrasonic transducer includes a concave-shaped emitting surface; the concave-shaped emitting surface includes a plurality of curve-shaped surfaces with different curvature radius, and the ultrasound wave is configured to form a three-dimensional focused area at at least two focused depths of human subcutaneous tissue through the emitting surface.

In an embodiment, a depth area of the focused area at least reaches a dermis layer of human subcutaneous tissue.

The present application further provides an ultrasonic treatment tip, applied to the ultrasonic treatment device as mentioned above. The ultrasonic treatment tip is provided with a treatment window, a liquid ultrasound-conducting medium is provided in the housing, and the ultrasonic treatment device further includes a ultrasound-transmitting membrane, and the ultrasound-transmitting membrane is provided at the treatment window to seal the treatment window and form an accommodating cavity for accommodating the liquid ultrasound-conducting medium.

In the technical solution of the present application, the ultrasonic treatment device with the large focused area includes a housing and an ultrasonic transducer installed inside the housing. Ultrasonic waves emitted by the ultrasonic transducer are configured to form a plurality of focused area units at one or more focused depths in a focused area, and each of the focused area units includes one or more of a focused point, a focused line, a focused plane or a focused volume. In this way, the energy in the focusing area can act evenly on the subcutaneous tissue layer of the user's target depth, thereby accurately treating the subcutaneous tissue layer of the target depth, and improving the treatment efficiency of the ultrasonic treatment device with the large focused area.

### BRIEF DESCRIPTION OF THE DRAWINGS

To more clearly illustrate technical solutions in the embodiments of the present application or the related art, the following will briefly introduce the drawings that need to be used in the description of the embodiments or the related art. Obviously, the drawings in the following description are only some embodiments of the present application. For those skilled in the art, without creative effort, other drawings can be obtained according to the structures shown in these drawings.

FIG. 1 is a schematic structural diagram of an ultrasonic treatment device with the large focused area according to an embodiment of the present application.

FIG. 2 is a schematic structural diagram of an ultrasonic transducer mechanism of the ultrasonic treatment device with the large focused area according to a first embodiment of the present application.

FIG. 3 is a schematic structural diagram of the ultrasonic transducer mechanism of the ultrasonic treatment device with the large focused area according to a second embodiment of the present application.

FIG. 4 is a schematic structural diagram of an acoustic lens of the ultrasonic treatment device with the large focused area according to an embodiment of the present application.

FIG. 5 is a schematic structural diagram of an ultrasonic treatment tip according to an embodiment of the present application.

FIG. 6 is a schematic structural diagram showing the structure being moved from the housing in FIG. 5.

FIG. 7 is a schematic diagram of the ultrasonic treatment tip of the present application after the ultrasound wave is emitted.

The realization of the objective, functional characteristics, and advantages of the present application are further described with reference to the accompanying drawings.

### DETAILED DESCRIPTION OF THE EMBODIMENTS

The technical solutions of the embodiments of the present application will be described in detail below with reference to the accompanying drawings. It is obvious that the embodiments described are only some rather than all of the embodiments of the present application. All other embodiments obtained by those skilled in the art based on the embodiments of the present application without creative efforts shall fall within the claimed scope of the present application.

It should be noted that all the directional indications (such as up, down, left, right, front, rear . . . ) in the embodiments of the present application are only used to explain the relative positional relationship, movement, or the like of the components in a certain posture (as shown in the drawings). If the specific posture changes, the directional indication will change accordingly.

Besides, the descriptions associated with, e.g., “first” and “second,” in the present application are merely for descriptive purposes, and cannot be understood as indicating or suggesting relative importance or impliedly indicating the number of the indicated technical feature. Therefore, the feature associated with “first” or “second” can expressly or impliedly include at least one such feature. In addition, the technical solutions of the various embodiments can be combined with each other, but the combinations must be based on the realization of those skilled in the art. When the combination of technical solutions is contradictory or cannot be achieved, it should be considered that such a combination of technical solutions does not exist, nor does it fall within the scope of the present application.

An ultrasonic treatment device with a large focused area is disclosed in the present application

The heat diffusion zone formed by the focused ultrasound waves in the subcutaneous tissue layer is generally in an ellipsoidal shape with the focused point as the center. In order to improve the treatment effect of each ultrasound wave and expand the effective area, the existing ultrasonic treatment device with the large focused area that has a single or more focused point to focus the ultrasound wave and form an ultrasound focused area disposed in the horizontal or vertical direction. The ultrasound focused area is formed under the subcutaneous tissue layer at the same depth or different depths, and the treatment effect on the subcutaneous tissue layer at different depths is also different, resulting in uneven distribution of energy required for treatment. In this way, in order to get better treatment for the subcutaneous tissue layer at the target depth, more ultrasound waves and time are needed for focused treatment, which seriously affects the treatment efficiency of the ultrasonic treatment device with the large focused area.

For this reason, the present application proposes an ultrasonic treatment device with the large focused area.

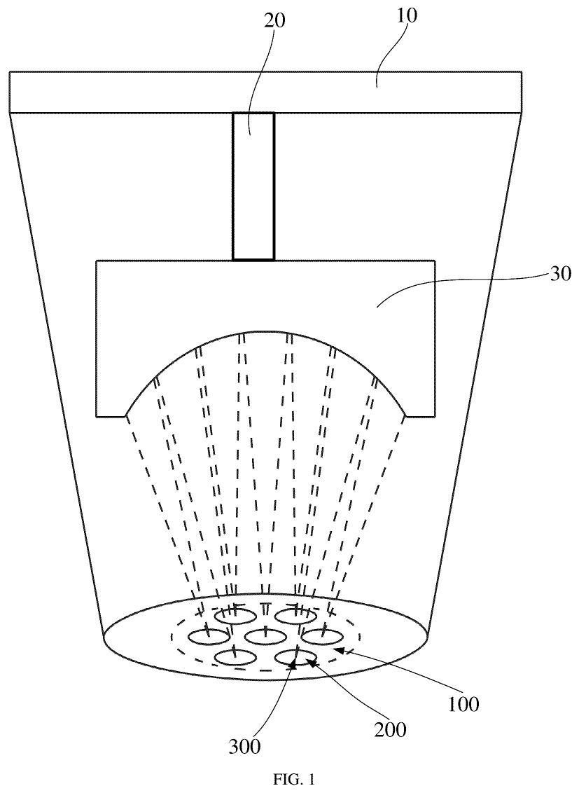

In an embodiment of the present application, as shown in FIG. 1 to FIG. 4, the ultrasonic treatment device with a large focused area includes a first housing 10, a first bracket 20 and an ultrasonic transducer mechanism 30. A treatment window is provided at one end of the first housing 10. The first bracket 20 is provided in the first housing 10. The ultrasonic transducer mechanism 30 is installed at the first bracket 20 and is opposite to the treatment window. The ultrasonic transducer mechanism 30 is configured to emit to the plane to be treated to form a first focused area 100 extending in the horizontal direction. The first focused area 100 includes a plurality of focused area units 200. Each focused area unit 200 includes one or more of a focused point 300 or a focused line. Understandable, each focused area unit 200 also includes a focused plane or a focused volume. The plurality of focused area units 200 are disposed at intervals.

In one embodiment, the ultrasonic transducer mechanism 30 is fixed inside the first housing 10 through the first bracket 20, and an emission port for emitting ultrasonic waves is provided at the ultrasonic transducer mechanism 30 and is opposite to the treatment window of the first housing 10. When the ultrasonic transducer mechanism 30 is powered on, the ultrasonic transducer mechanism 30 will transmit ultrasonic waves to the target depth underneath the user's skin (the target plane needing to be treated) through the treatment window of the first housing 10, and the ultrasonic waves emitted by the ultrasonic transducer mechanism 30 will form a three-dimensional heat diffusion zone in the user's subcutaneous tissue layer. The projection of the three-dimensional heat diffusion zone vertically on the user's skin is a first focused area 100 extending in the horizontal direction, and the first focused area 100 is formed by a plurality of focused area units 200 disposed and spliced in sequence in the horizontal direction, so that the ultrasonic waves emitted by the ultrasonic transducer mechanism 30 can accurately act on the subcutaneous tissue layer at the user's target depth, and will not enter the subcutaneous tissue layer at the non-target depth that does not require treatment.

When each focused point 300 acts on the subcutaneous tissue layer at the target depth, each focused point 300 has a corresponding heat diffusion zone, and the heat diffusion zone has heat that diffuses outwards. The area of the projection of the heat diffusion zone for each focused point 300 perpendicular to the plane to be treated is the focused area unit 200. Therefore, by distributing a plurality of focused points 300 at intervals, the heat diffusion zones corresponding to two adjacent focused points 300 will not overlap, so that each focused area unit 200 can act on the subcutaneous tissue layer at the same target depth respectively, which is beneficial for the ultrasound energy to be focused on an accurate tissue treated more precisely at a certain depth under the skin. In this way, the energy used by the first focused area 100 can act evenly on the subcutaneous tissue layer at the target depth of the user, thereby accurately treating the subcutaneous tissue layer at the target depth, and improving the treatment efficiency of the ultrasonic treatment device with the large focused area.

In one embodiment, as shown in FIG. 1 to FIG. 4, a plurality of focused points 300 or focused lines are disposed in a rectangular array, a circular array, an elliptical array, or an annular array. The first focused area 100 formed by the plurality of focused points 300 is also disposed in a rectangular shape, a circular shape, an elliptical shape or an annular shape, and the shape of the first focused area 100 is more regular. In this way, not only the operating personnel can control the treatment area more accurately when operating the ultrasonic treatment device with the large focused area, but also the operating personnel can calculate the area of the user's treatment area conveniently, to make accurate data statistics and reference for subsequent treatment plans.

In another embodiment, the plurality of focused points 300 can also be disposed both in a circular array and an annular array. Or, the plurality of focused points 300 can also be disposed in a rectangular array or in an annular array, and the like, and the specific array arrangement combinations are not listed here.

By using a variety of shape arrangement combinations together, the first focused area 100 formed by the plurality of focused points 300 can form an irregular shape, which is more conductive for treating the user's irregular treatment area, thereby increasing the treatment area of the ultrasonic treatment device with the large focused area.

In one embodiment, as shown in FIG. 1 to FIG. 4, each focused point 300 is provided with a corresponding heat diffusion area.

In an embodiment, the focused ultrasonic energy will show a thermal effect in the subcutaneous tissue layer of the human body, and heat will diffuse around the focused point 300, that is, the temperature in the center is highest and will gradually decrease in the radial direction. The area where the temperature of the thermal effect can achieve the effective treatment is called the thermal diffusion zone, and the height of the thermal diffusion zone is the height of the space in which the focused point 300, the focused line, the focused plane or the focused volume disposed in the subcutaneous tissue layer along the horizontal or vertical direction. Generally, by making the height of each thermal diffusion zone being equal to the thickness of the focused area unit 200, the single-shot ultrasonic energy corresponding to each focused point 300 can fully act on the subcutaneous tissue layer of the human body, thereby improving the energy utilization rate of the focused area unit 200, improving the treatment effect of the ultrasonic treatment device with the large focused area.

In one embodiment, as shown in FIG. 1 to FIG. 4, the central temperature of all the focused points 300 is the same, and the central temperature of each focused point 300 is in the area from 48° C. to 80° C.

In one embodiment, the central temperature of the focused point 300 fall within this area, and the thermal diffusion zone corresponding to the focused point 300 can not only avoid damaging the user's skin, but also achieve an effective treatment effect. The central temperature of the focused point 300 is determined by the specific energy value and the focused distance of a single ultrasonic wave, and the specific energy value and focused distance of a single ultrasonic wave can be calculated by the main control chip of the ultrasonic transducer mechanism 30, and the operation is controlled by the main control chip, so that the ultrasonic transducer mechanism 30 can accurately treat the user.

In one embodiment, as shown in FIG. 1 to FIG. 4, the central temperatures of two adjacent focused points 300 are set differently. In this way, each focused point 300 can be set according to the actual treatment plan of the user, that is, the position with high treatment intensity is provided with a focused point 300 with a higher central temperature for treatment, and the position with low treatment intensity is provided with a focused point 300 with a lower central temperature for treatment, thereby improving the treatment efficiency of the ultrasonic treatment device with the large focused area.

In one embodiment, as shown in FIG. 1 to FIG. 4, each focused area unit 200 is in a rectangular shape, a circular shape, an elliptical shape or an annular shape.

In one embodiment, by setting the first emitting surface 31 of different shapes on the transducer or setting the emitting port of different shapes for the emitting tip of the transducer, the focused area unit 200 can be arranged in different shapes. A single ultrasonic wave is emitted by the transducer and forms a three-dimensional heat diffusion area in the subcutaneous tissue layer of the user. The projection of the heat diffusion area of the sphere vertical to the skin surface of the user is a circular focused area unit 200. The projection of the heat diffusion area of the rectangle vertical to the skin surface of the user is a rectangular focused area unit 200. The projection of the heat diffusion area of the ellipse vertical to the skin surface of the user is an ellipsoidal focused area unit 200. The projection of the heat diffusion area of the annulus vertical to the skin surface of the user is an annular focused area unit 200.

The shape of the focused area unit 200 to be a regular shape, such as a rectangular shape, a circular shape, or an ellipsoidal shape, which allows the operating personnel to control the treatment area more conveniently, that is, on the same plane to be treated, as long as the plurality of focused area units 200 are disposed in sequence in the horizontal direction, there will be no overlapping positions between the two adjacent focused area units 200, thereby improving the treatment efficiency of the ultrasonic treatment device with the large focused area.

In one embodiment, as shown in FIG. 1 to FIG. 4, shapes of the plurality of focused area units 200 are set in the same manner. Each focused area unit 200 is in the same shape, so that not only the plurality of focused area units 200 can form a regular first focused area 100, but also the first focused area 100 after splicing is more uniform, and the gap between the two adjacent focused area units 200 is smaller, thereby improving the treatment effect of the ultrasonic treatment device with the large focused area.

In another embodiment, as shown in FIG. 1 to FIG. 4, the shapes of the plurality of focused area units 200 are set in different ways. The focused area units 200 of different shapes are combined and disposed according to the actual treatment area and scheme of the current user. In this way, the first focused area 100 formed by the focused area units 200 of different shapes can treat the user more specifically, thereby ensuring the effect of ultrasonic treatment.

In the first embodiment, as shown in FIG. 1 to FIG. 4, the ultrasonic transducer mechanism 30 includes a plurality of transducers provided at the first bracket 20, and the plurality of transducers are disposed at intervals. Each transducer is configured to form a focused point 300. By fixing a plurality of transducers emitting single-shot ultrasonic energy to the first bracket 20, each transducer can form a focused point 300. Compared with the previous method of focusing with a single transducer at the same frequency, in this embodiment, a plurality of transducers emitting low-frequency ultrasonic energy are used for treatment. The plane first focused area 100 formed by the ultrasonic transducer mechanism 30 of this structure can provide more uniform energy, which can perform effective and quick treatment without harming the user's skin, thereby improving the treatment effect of the ultrasonic transducer mechanism 30.

In the second embodiment, as shown in FIG. 1 to FIG. 4, the ultrasonic transducer mechanism 30 is a transducer disposed on the first bracket 20, and the transducer is provided with a plurality of first emitting surfaces 31. The curvature regions corresponding to the plurality of first emitting surfaces 31 are different, and each curvature region corresponds to a focused point 300. In an embodiment, in view of the different distances between the plurality of first emitting surfaces 31 and the user's skin, a plurality of first emitting surfaces 31 are provided at a single transducer with different curvatures, so that the first emitting surfaces 31 with different curvatures can form a focused point 300 with equal energy in the user's subcutaneous tissue layer, and then form a plane first focused area 100 with uniform energy distribution, thereby improving the treatment effect of the ultrasonic transducer mechanism 30.

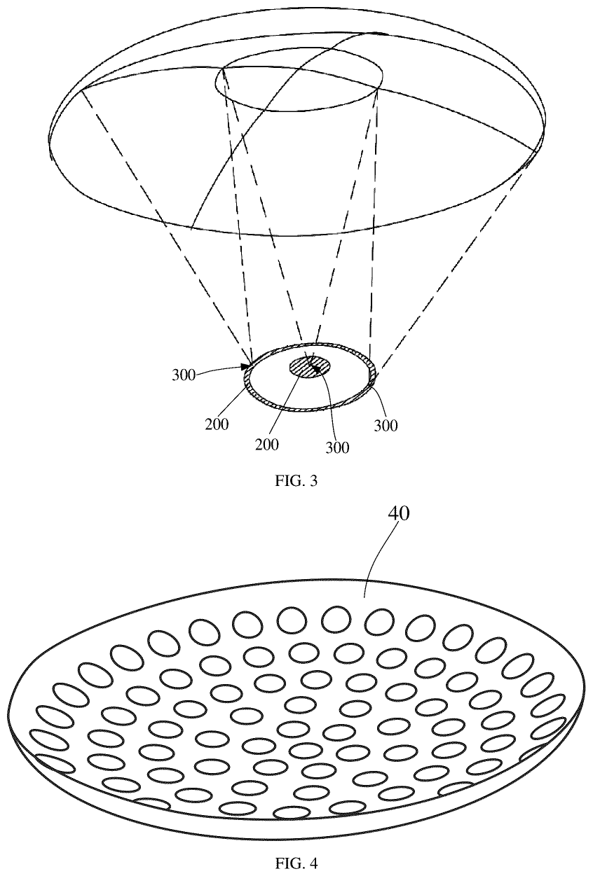

In one embodiment, as shown in FIG. 3, the ultrasonic transducer mechanism 30 includes a central transducer and at least one sub-transducer. The sub-transducer is disposed adjacent to the central transducer and is located outside the central transducer. The central transducer is a spherical transducer, and the sub-transducer is an annular transducer.

In one embodiment, more types of combined shapes of focused areas can be formed by combining the central transducer and the sub-transducer. In this way, more types of combined shapes of focused areas are more suitable for complex treatment needs of different users, thereby increasing the treatment area of the ultrasonic treatment device with the large focused area. In this embodiment, the central transducer can be a spherical transducer, and the sub-transducer can be an annular transducer. In this way, the spherical transducer in the center forms a circular focused area on the skin surface, and the annular transducer forms an annular focused area on the skin surface. The circular focused area and the annular focused area are combined. The shapes of the central transducer and the sub-transducer are not limited to the above shapes, but can also be other shapes as long as they meet the actual treatment requirements.

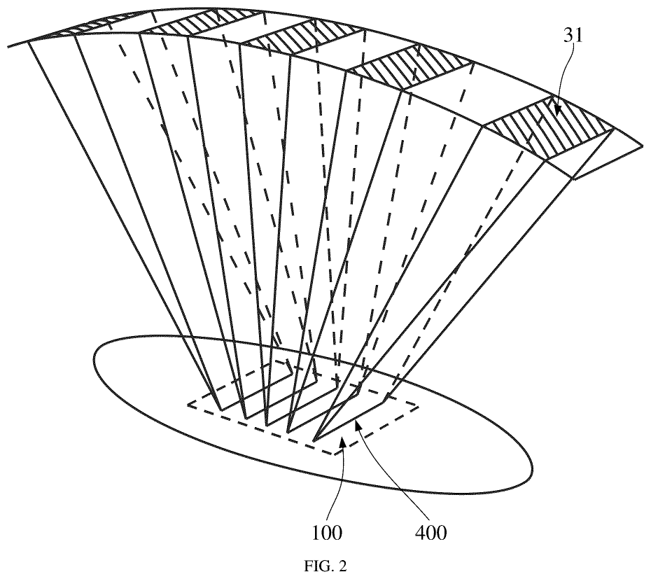

In one embodiment, as shown in FIG. 2, the transducer is a tile-shaped transducer, and the tile-shaped transducer is configured to focus to form a focused line 400. The tile-shaped transducer is focused on the subcutaneous tissue layer of the user, and the projection of the heat diffusion zone focused by the tile-shaped transducer is a line (focused line 400) vertical on the plane to be treated, and a plurality of focused lines 400 are located on the same plane, thereby converging a first focused area 100 shaped in a rectangular plane. The converged plane first focused area 100 is easier to be flattened on the plane to be treated, and the energy of the plane first focused area 100 is more uniform, thereby improving the treatment effect of the ultrasonic treatment device with the large focused area.

In one embodiment, as shown in FIG. 1, a plurality of focused points 300 are disposed at equal intervals. The interval between two adjacent focused points 300 ranges from 0.1 mm to 2 mm.

In one embodiment, a plurality of focused points 300 are disposed at the same interval, so that the two focused area units 200 corresponding to the two adjacent focused points 300 will not overlap, which not only makes the energy corresponding to the first focused area 100 more uniform, but also facilitates the control on the energy formed by the first focused area 100. The specific distance between two adjacent focused points 300 is determined by the position to be treated or the treatment needs of the user, such that the heat diffusive zones generated by the plurality of the focused points 300 are adjacent with each other, which is beneficial to improve the uniformity of ultrasound energy in the focused area under the skin, finally the treatment effect is improved. For example, if the position to be treated is the arm, the distance between the two focused points 300 will be smaller, so that energy formed by the first focused area 100 is relatively concentrated, and the effect on the skin of the arm with a thicker subcutaneous fat layer is more efficient. If the position to be treated is the face, the distance between the two focused points 300 will be larger, so that energy formed by the focus is relatively dispersed, and the effect on the skin of the face with a thinner subcutaneous fat layer is more delicate, thereby reducing the damage to the skin of the face.

In one embodiment, as shown in FIG. 1 and FIG. 4, the ultrasonic treatment device with the large focused area also includes an acoustic lens 40, which is provided at the ultrasonic transducer mechanism 30 and is configured to gather the ultrasonic waves emitted by the ultrasonic transducer mechanism 30. The acoustic lens 40 is a curve-shaped acoustic lens 40, and the acoustic lens 40 is installed at the first emitting surface 31 of the transducer. A curve-shaped acoustic lens 40 is installed at the first emitting surface 31. After the first emitting surface 31 emits ultrasound waves, the ultrasound waves are refracted in the curve-shaped acoustic lens 40 to form a first focused area 100 with more uniform and stable energy outside the treatment window, to treat the user, thereby improving the treatment effect of the ultrasonic treatment device with the large focused area.

It is should be noted that in the above-mentioned transducer, when the first emitting surface 31 is a curve-shaped surface, the user can be treated by ultrasound waves emitted from the curve-shaped surface. When the first emitting surface 31 is plane-shaped, a curve-shaped acoustic lens 40 is required to focus the ultrasonic wave beam, so that when the first emitting surface 31 emits ultrasound waves, the ultrasound waves are refracted in the curve-shaped acoustic lens 40 to treat the user.

An ultrasonic treatment tip and an ultrasonic treatment device are disclosed in the present application.

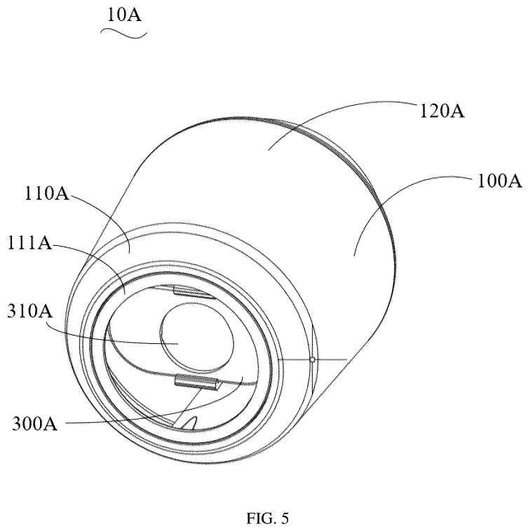

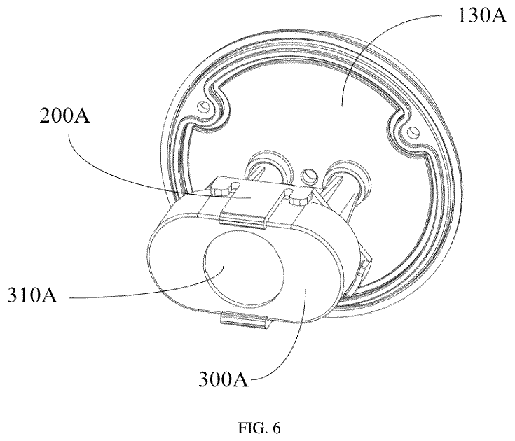

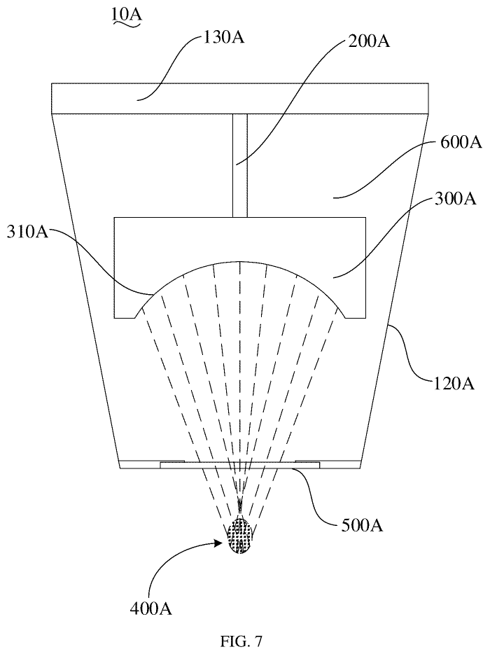

As shown in FIG. 5 to FIG. 7, in one embodiment of the present application, the ultrasonic treatment tip 10A includes a second housing 100A, a second bracket 200A and an ultrasonic transduction unit 300A. One end of the second housing 100A is provided with a treatment window 111A, and the second bracket 200A is provided in the second housing 100A. The ultrasonic transduction unit 300A is installed at the second bracket 200A, and the ultrasonic transduction unit 300A is provided with a second emitting surface 310A. The ultrasonic waves emitted by the second emitting surface 310A form a plurality of focused points outside the treatment window 111A, and the plurality of focused points converge into a second focused area 400A.

The cross section of the second housing 100A can be in a rectangular shape, a circular shape, a polygonal shape, a combined shape, or an irregular shape, which can be selected and designed according to actual use requirements, and will not be limited here.

One end of the second housing 100A is configured as a treatment end 110A, and the treatment window 111A is provided at the treatment end 110A. The treatment end 110A is configured to contact the skin surface of the human body. The second bracket 200A is mainly configured to fix and install the ultrasonic transduction unit 300A. The second bracket 200A is made of the hard material. As for the type of the hard material, it can be a hard material such as ABS, HIPS, PP, PC, or a metal or the alloy material, and the like, which will not be limited here. The second emitting surface 310A of the ultrasonic transduction unit 300A is provided toward the treatment end 110A. When the ultrasonic treatment is performed, the second emitting surface 310A of the ultrasonic transduction unit 300A will emit ultrasonic waves, and ultrasonic waves will pass through the treatment window 111A at the treatment end 110A. Then ultrasonic waves will enter the action site of the subcutaneous tissue after passing through the skin surface of the human body to take effect on the subcutaneous tissue. The shape of the treatment window 111A can be a circle, or can be a square, or can be a prism, which will not be limited here.

The second housing 100A includes a housing 120A and a base 130A. The housing 120A is connected to the base 130A, and the second bracket 200A is installed at the base 130A. The housing 120A and the base 130A are enclosed to form a closed accommodating cavity for installing the ultrasonic transduction unit 300A and the second bracket 200A. The second bracket 200A is fixedly installed at the base 130A, and the ultrasonic transduction unit 300A is fixedly installed at the second bracket 200A, which will further increase the stability of the ultrasonic transduction unit 300A.

The curvature of the second emitting surface 310A is different, so that the plurality of focused points emitted by the second emitting surface 310A will converge into a second focused area 400A, and the second focused area 400A is in a three-dimensional shape. Through the second focused area 400A, the user is treated, and the treatment area is larger. Compared with the conventional point treatment, the second focused area 400A can provide higher treatment efficiency, and the energy of the second focused area 400A is more uniform, and the treatment effect is better.

The ultrasonic treatment tip 10A of the present application includes a second housing 100A, a second bracket 200A and an ultrasonic transduction unit 300A. A treatment window 111A is provided at one end of the second housing 100A. The second bracket 200A is provided in the second housing 100A. The ultrasonic transduction unit 300A is installed at the second bracket 200A, and the ultrasonic transduction unit 300A is provided with a second emitting surface 310A. The second emitting surface 310A is provided with a plurality of focused points outside the treatment window 111A, and the plurality of focused points converge into a second focused area 400A. In this way, the user is treated through the second focused area 400A, the treatment area is larger, and compared with the conventional point treatment, the second focused area 400A can provide a higher treatment efficiency. The energy of the second focused area 400A is more uniform, and the treatment effect is better.

As shown in FIG. 5 to FIG. 7, in one embodiment, the second emitting surface 310A is a curve-shaped surface. The second emitting surface 310A can be a curve-shaped surface, and ultrasound waves are emitted through the curve-shaped second emitting surface 310A. A second focused area 400A is formed outside the treatment window 111A to treat the user.

In another embodiment, the second emitting surface 310A is plane-shaped, and the ultrasonic treatment tip 10A further includes an acoustic lens, which is a curve-shaped acoustic lens, and the acoustic lens is installed at the second emitting surface 310A. The second emitting surface 310A can also be plane-shaped, and a curve-shaped acoustic lens is installed at the second emitting surface 310A. After the second emitting surface 310A emits ultrasound waves, the ultrasound waves are refracted in the curve-shaped acoustic lens to form a second focused area 400A outside the treatment window 111A, thereby treating the user.

In one embodiment, the second focused area 400A is shaped in a sphere,
or the second focused area 400A is shaped in an ellipsoid. In an embodiment, the second focused area 400A is in a three-dimensional shape. Compared with a point or a surface, the second focused area 400A shaped in a sphere or an ellipsoid can provide a larger area, which can achieve better treatment effects and improve treatment efficiency.

The ultrasonic transduction unit 300A of the present application is provided with a second emitting surface 310A, and the second emitting surface 310A is provided with a plurality of focused points outside the treatment window 111A. The plurality of focused points converge into a second focused area 400A. The second focused area 400A corresponds to a plurality of depths, so that the ultrasonic transduction unit 300A can provide a treatment effect at a plurality of depths. Therefore, the ultrasonic transduction unit 300A can simultaneously enter the human subcutaneous tissue in different depth areas. When the ultrasonic treatment tip 10A is working, the ultrasonic treatment tip 10A can simultaneously act on the human subcutaneous tissue at different depths, to achieve the purpose of treatment, thereby further meeting the different needs of users.

In one embodiment, the depth of the second focused area 400A is in an area from 0.5 mm to 2.5 mm. In an embodiment, the depth of the ultrasonic transduction unit 300A entering the subcutaneous tissue of the human body can range from 0.5 mm to 2.5 mm, which is the dermis layer of the human body. When the ultrasonic treatment tip 10A is working, the ultrasonic treatment tip 10A can cause mechanical effects and thermal effects, resulting in immediate contraction of collagen, causing inflammatory effects, and promoting collagen regeneration. Within this depth area, the ultrasonic treatment tip 10A is mainly used for the treatment of skin firming and wrinkles.

In one embodiment, the depth of the second focused area 400A ranges from 1.5 mm to 3.5 mm. In an embodiment, the depth of the ultrasonic transduction unit 300A entering the subcutaneous tissue of the human body can also range from 1.5 mm to 3.5 mm, which is the subcutaneous fat layer of the human body. When the ultrasonic treatment tip 10A is working, the ultrasonic treatment tip 10A can inactivate fat cells, cause differentiation of adipose stem cells to be differentiated into fibroblasts, and promote tissue firmness and density. Within this depth area, the ultrasonic treatment tip 10A is mainly used to treat atrophic scars, obesity and other clinical problems.

In one embodiment, the depth of the second focused area 400A can range from 3 mm to 15 mm. In an embodiment, the depth of the ultrasonic transduction unit 300A entering the subcutaneous tissue of the human body can also range from 3 mm to 15 mm, which is the subcutaneous fascia layer of the human body. When the ultrasonic treatment tip 10A is working, the ultrasonic treatment tip 10A can tighten the skin, improve the body contour, and lift the muscle base to achieve tightening and lifting.

It should be noted that the depth of the ultrasonic transduction unit 300A entering the subcutaneous tissue of the human body can also be in other areas, including but not limited to the above three areas, which will not be limited here.

In one embodiment, the operating frequency of the ultrasonic transduction unit 300A can be in the area from 0.5 MHz to 10 MHz.

In an embodiment, when the ultrasonic transduction unit 300A enters the subcutaneous tissue of the human body at a depth area from 0.5 mm to 2.5 mm, the depth is the dermis of the human body. In this case, the frequency of the ultrasonic treatment tip 10A ranges from 4 MHz to 10 MHz, and the power ranges from 1 W to 10 W. When the ultrasonic treatment tip 10A is working, the ultrasonic treatment tip 10A can cause mechanical effects and thermal effects, resulting in immediate contraction of collagen, causing inflammatory effects, and promoting collagen regeneration. Within this depth area, the ultrasonic treatment tip 10A is mainly used for the treatment of skin firming, wrinkles, and the like.

When the ultrasonic transduction unit 300A enters the subcutaneous tissue of the human body at a depth area from 1.5 mm to 3.5 mm, the depth is the subcutaneous fat layer of the human body. In this case, the frequency of the ultrasonic treatment tip 10A ranges from 1 MHz to 4 MHZ, and the power ranges from 3 W to 40 W. When the ultrasonic treatment tip 10A is working, the ultrasonic treatment tip 10A can inactivate fat cells, cause the differentiation of fat stem cells to be differentiated into fibroblasts, and promote the firmness and density of tissues. Within this depth area, the ultrasonic treatment tip 10A is mainly used to treat clinical problems, such as atrophic scars and obesity.

When the ultrasonic transduction unit 300A enters the subcutaneous tissue of the human body at a depth area from 3 mm to 15 mm, the depth is the subcutaneous fascia layer of the human body. In this case, the frequency of the ultrasonic treatment tip 10A ranges from 0.5 MHz to 2 MHz, and the power ranges from 3 W to 40 W. When the ultrasonic treatment tip 10A is working, the ultrasonic treatment tip 10A can tighten the skin, improve the body contour, and lift the muscle base to achieve firmness and lifting.

As shown in FIG. 5 to FIG. 7, in one embodiment, a liquid ultrasound-conducting medium is provided in the second housing 100A, and the ultrasonic treatment tip 10A further includes a ultrasound-transmitting membrane 500A, which is provided at the treatment window 111A to seal the treatment window 111A and form an accommodating cavity 600A for accommodating the liquid ultrasound-conducting medium. In an embodiment, the ultrasonic wave mainly enters the subcutaneous layer of the human body through the treatment window 111A. Since the conduction of the ultrasonic wave requires the liquid medium, a liquid ultrasound-conducting medium is also provided in the second housing 100A to transmit the ultrasonic waves emitted by the ultrasonic treatment tip through the liquid ultrasound-conducting medium. The ultrasound-transmitting membrane 500A is provided at the treatment window 111A, and the ultrasonic wave will first pass through the ultrasound-transmitting membrane 500A and then reach the human skin, thereby treating the patient. At the same time, the treatment window 111A is sealed by the ultrasound-transmitting membrane 500A to avoid the leakage of the liquid ultrasound-conducting medium.

The present application further provides an ultrasonic treatment device, which includes an ultrasonic treatment tip 10A. The specific structure of the ultrasonic treatment tip 10A may refer to the above embodiments. Since the ultrasonic treatment device adopts all the technical solutions of the above embodiments, it has at least all the beneficial effects brought by the technical solutions of the above embodiments, which will not be repeated here.

The above are only some embodiments of the present application, and do not limit the scope of the present application thereto. Under the concept of the present application, any equivalent structural transformation made according to the description and drawings of the present application, or direct/indirect application in other related technical fields shall fall within the claimed scope of the present application.

What is claimed is:

1. An ultrasonic treatment device with a large focused area, comprising:
a housing; and
an ultrasonic transducer installed inside the housing, wherein ultrasonic waves emitted by the ultrasonic transducer are configured to form a plurality of focused area units at one or more focused depths in a focused area, and each of the focused area units comprises one or more of a focused point, a focused line, a focused plane or a focused volume.

2. The ultrasonic treatment device according to claim 1, wherein the plurality of focused area units are disposed in a rectangular array, a circular array, an elliptical array or a circular array.

3. The ultrasonic treatment device according to claim 2, wherein each of the focused point, the focused line and the focused volume has a corresponding heat diffusion zone.

4. The ultrasonic treatment device according to claim 3, wherein central temperatures of the plurality of focused area units are the same, and the central temperature of each focused area unit is in an area from 48° C. to 80° C.

5. The ultrasonic treatment device according to claim 3, wherein central temperatures of two adjacent focused area units are different.

6. The ultrasonic treatment device according to claim 1, wherein shapes of the plurality of focused areas are the same, and each focused area is in a rectangular shape, a circular shape, an elliptical shape or an annular shape.

7. The ultrasonic treatment device according to claim 1, wherein shapes of the plurality of focused area units are different, and each focused area is in a rectangular shape, a circular shape, an elliptical shape or an annular shape.

8. The ultrasonic treatment device according to claim 1, wherein:
the ultrasonic transducer comprises a concave-shaped emitting surface for focusing an ultrasonic wave beam, and
the concave-shaped emitting surface comprises a plurality of emitting surface units disposed in an interval array; the plurality of emitting surface units are disposed in a concave array, and the plurality of emitting surface units have different curvature radius.

9. The ultrasonic treatment device according to claim 1, wherein the ultrasonic transducer comprises a plurality of sub-transducer arrays, and the plurality of sub-transducers comprise a plurality of different focused depths, and are configured to form a plurality of focused area units at at least two focused depths.

10. The ultrasonic treatment device according to claim 1, wherein the ultrasonic transducer comprises a central transducer and at least one sub-transducer, and the sub-transducer is provided adjacent to the central transducer and is provided outside the central transducer; the central transducer is a spherical transducer, and the sub-transducer is an annular transducer.

11. The ultrasonic treatment device according to claim 9, wherein the plurality of sub-transducers comprise a plurality of tile-shaped transducers disposed in an array; the plurality of tile-shaped transducers are configured to form a plurality of focused area units the same focused depth, and the focused area units comprise focused lines.

12. The ultrasonic treatment device according to claim 1, wherein the plurality of focused area units are disposed at equal intervals, and/or a distance between two adjacent focused areas are in an area from 0.1 mm to 2 mm.

13. The ultrasonic treatment device according to claim 1, wherein the ultrasonic transducer comprises a plane-shaped emitting surface and a concave-shaped acoustic lens for focusing an ultrasonic wave beam; the acoustic lens comprises small lenses with different curvature radius, and the small lenses are independent with each other.

14. The ultrasonic treatment device according to claim 1, wherein the ultrasonic transducer comprises an emitting surface and an ultrasonic treatment tip, and the emitting surface is plane-shaped; the ultrasonic treatment tip further comprises an acoustic lens, and the acoustic lens is a curve-shaped acoustic lens; the acoustic lens is installed at the emitting surface, and an ultrasound wave is refracted from the curve-shaped acoustic lens at at least two focused depths of human subcutaneous tissue to form a three-dimensional focused area.

15. The ultrasonic treatment device according to claim 1, wherein the ultrasonic transducer comprises a concave-shaped emitting surface; the concave-shaped emitting surface comprises a plurality of curve-shaped surfaces with different curvature radius, and the ultrasound wave is configured to form a three-dimensional focused area at at least two focused depths of human subcutaneous tissue through the emitting surface.

16. The ultrasonic treatment device according to claim 1, wherein a depth area of the focused area at least reaches a dermis layer of human subcutaneous tissue.

17. The ultrasonic treatment device according to claim 1, wherein the ultrasonic waves emitted by the ultrasonic transducer are configured to form a plurality of focused area units at the same focused depth in the focused area that is shaped as the focused plane.

18. An ultrasonic treatment tip, applied to the ultrasonic treatment device according to claim 1, wherein the ultrasonic treatment tip comprises:
a housing; and
an ultrasonic transducer installed inside the housing, wherein ultrasonic waves emitted by the ultrasonic transducer are configured to form a plurality of focused area units at one or more focused depths in a focused area, and each of the focused area units comprises one or more of a focused point, a focused line, a focused plane or a focused volume;
wherein the treatment tip is provided with a treatment window, a liquid ultrasound-conducting medium is provided in the housing, and the ultrasonic treatment device further comprises an ultrasound-transmitting membrane, and the ultrasound-transmitting membrane is provided at the treatment window to seal the treatment window and form an accommodating cavity for accommodating the liquid ultrasound-conducting medium.
